(19) United States
(12) Patent Application Publication
(10) Pub. No.: US 2019/0083304 A1
(43) Pub. Date: Mar. 21, 2019

(54) SYSTEMS AND METHODS FOR COMBINED FEMTO-PHACO SURGERY

(71) Applicant: Doug Patton, Irvine, CA (US)

(72) Inventor: Doug Patton, Irvine, CA (US)

(21) Appl. No.: 15/791,385

(22) Filed: Oct. 23, 2017

Related U.S. Application Data
(60) Provisional application No. 62/411,441, filed on Oct. 21, 2016.

(51) Int. Cl. A61F 9/008; A61B 34/00; A61F 9/007; A61B 50/33

(52) U.S. Cl. CPC A61F 9/008; A61B 34/25; A61B 90/25; A61F 9/00781; A61B 50/33; A61F 9/00736

(57) ABSTRACT

Systems and methods are described for a combined femto/phaco surgical system built into a single housing with at least one component that can articulate into and out of position when needed. Preferred systems include femto and phaco surgical systems that can be

This application claims priority to U.S. provisional application having Ser. No. 62/411,441 filed on Oct. 21, 2016. This and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is Femto-Phaco Surgery.

### BACKGROUND

The following description includes information that may be useful in understanding the present invention. It is not an admission that any of the information provided herein is prior art or relevant to the presently claimed invention, or that any publication specifically or implicitly referenced is prior art.

FIG. 1 illustrates various examples of surgical environments for Phaco Cataract surgery.

FIG. 2 illustrates various examples of surgical environments for Femto Lasik surgery.

FIG. 3 illustrates various examples of surgical environments for Femto Cataract surgery.

The problem: currently a patient has to be wheeled from one operating room to the other during the surgery between the Femto Laser and Phaco machine. This is obviously not good for the patient. In addition, Applicant is unaware of any shared procedural patient data information shared between the two machines which does not allow a surgeon to build on previous surgery data or combine procedures. In addition, there exists is no way to easily go between the two machines during surgery for detail patient modifications.

Although great work has been done to address these problems, such as described in U.S. Pat. No. 8,986,290 to Patton, more work needs to be done to improve the patient outcome and eliminate the above problems.

Thus, there is still a need for an all-in-one machine that can eliminate the need to move patients during procedures and that allows for the sharing of patient data information.

### SUMMARY OF THE INVENTION

The inventive subject matter provides apparatus, systems and methods for a combined Femto-Phaco surgical apparatus. The core of the system is the swivel technology that allows one to easily articulate radially the Femto and Phaco arms into position during surgery. The arms not required articulate over the main housing.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 illustrates various examples of surgical environments for Phaco Cataract surgery.

FIG. 2 illustrates various examples of surgical environments for Femto Lasik surgery.

FIG. 3 illustrates various examples of surgical environments for Femto Cataract surgery.

FIGS. 4-7 illustrate various views of one embodiment of the inventive subject matter in a femto arrangement.

FIGS. 8-9 illustrate various views of one embodiment of the inventive subject matter in a phaco arrangement.

FIG. 10 illustrates top views showing the femto and phaco system set up.

FIG. 11 illustrates a top view showing the femto arrangement.

FIG. 12 illustrates a perspective view of the system in a phaco arrangement.

FIGS. 13-18 are various views of the device of FIG. 12 in the phaco arrangement.

FIG. 19 is an exemplary embodiment of the graphical user interface for the phaco arrangement.

FIG. 20 is an exemplary embodiment of the graphical user interface for the femto arrangement.

FIG. 21 is an exemplary embodiment of the graphical user interface for the combined phaco/femto setup.

FIGS. 22-23 are schematics of one embodiment of the phaco/femto system.

FIGS. 24-39 illustrate various views of another embodiment of the phaco/femto system shown in line drawings.

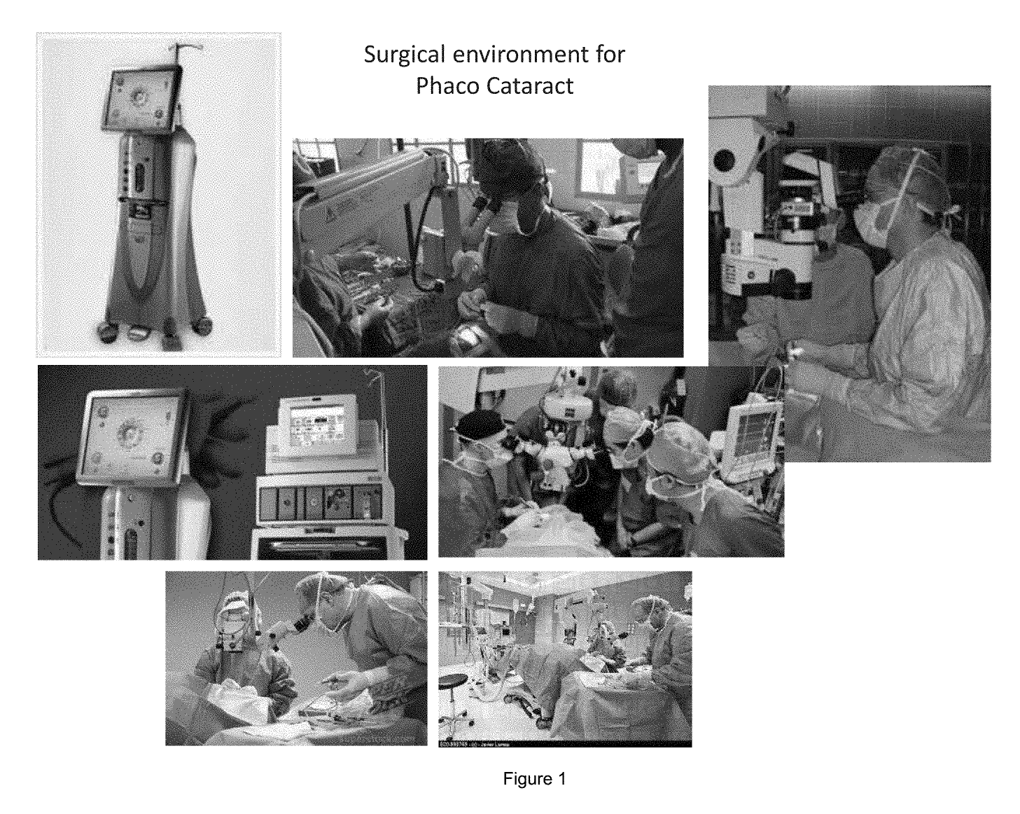

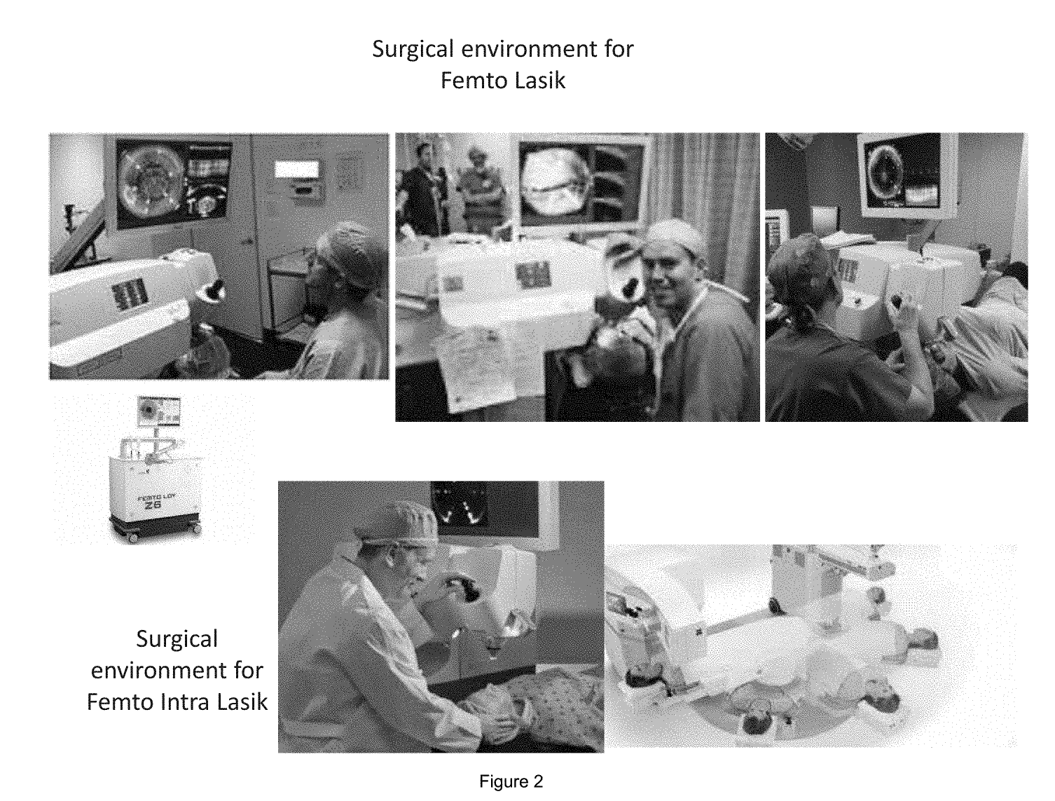

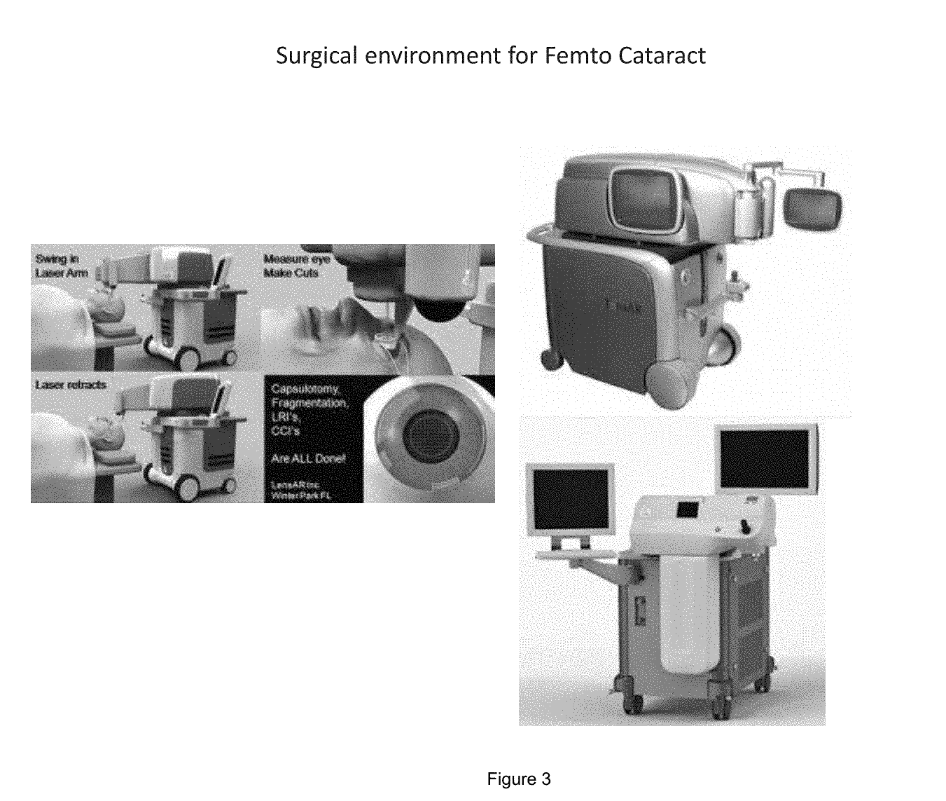

### DETAILED DESCRIPTION

Throughout the following discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

This combination phaco femto cataract product will be the future of ophthalmology, every surgery can benefit from the systems and methods described herein.

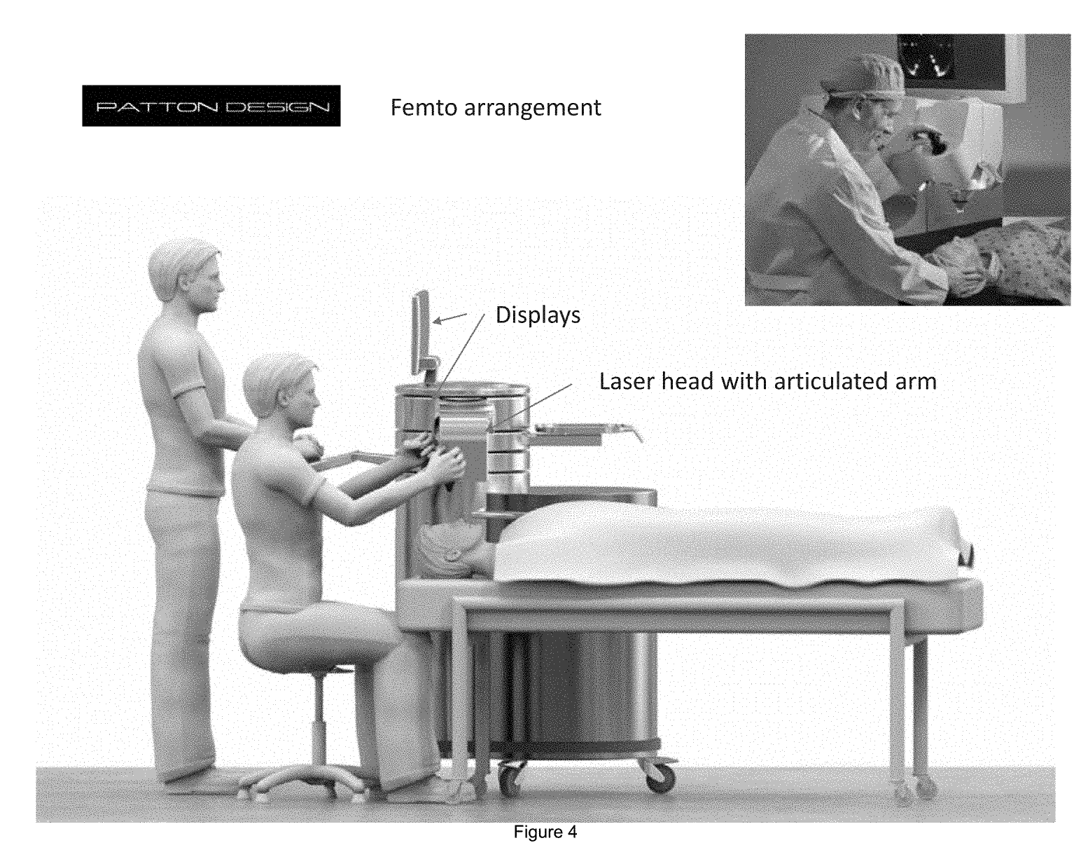

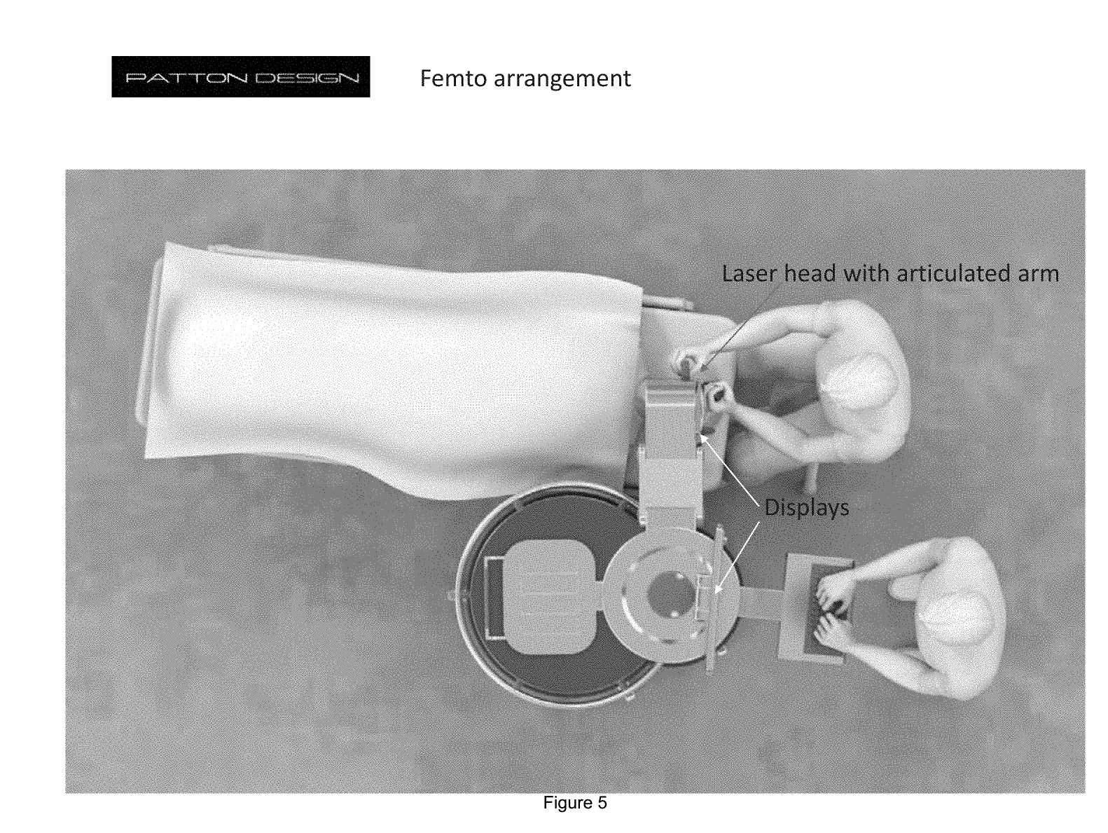

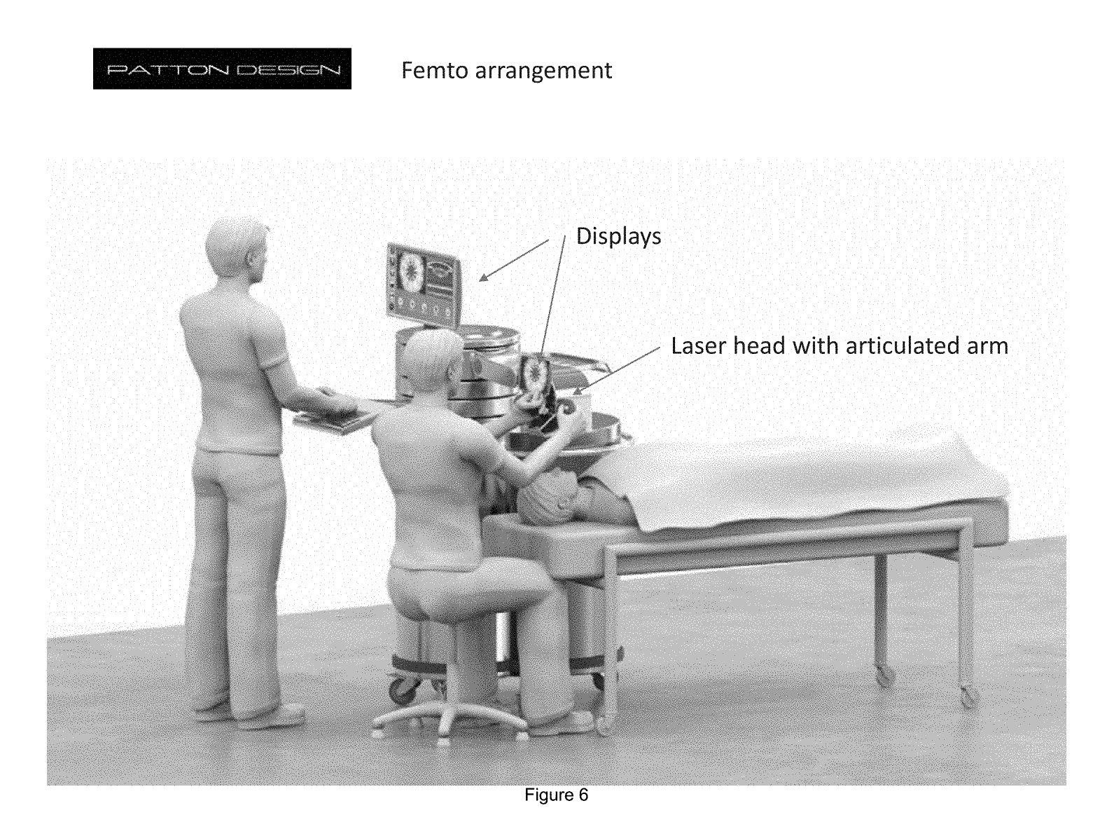

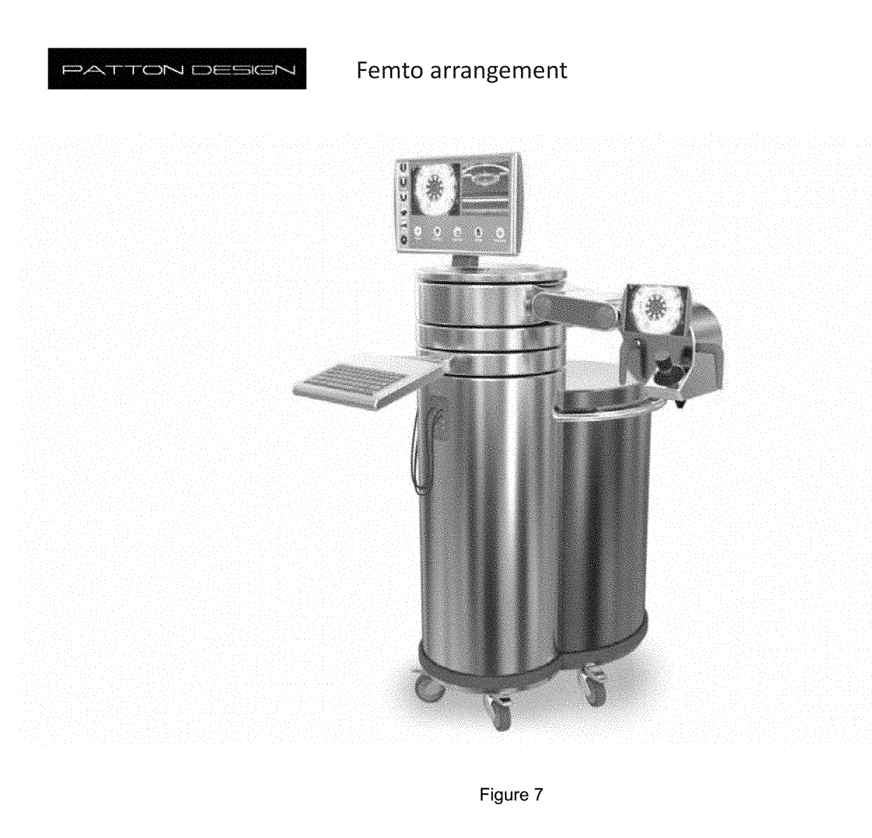

FIGS. 4-7 illustrate various views of one embodiment of the inventive subject matter in a femto arrangement.

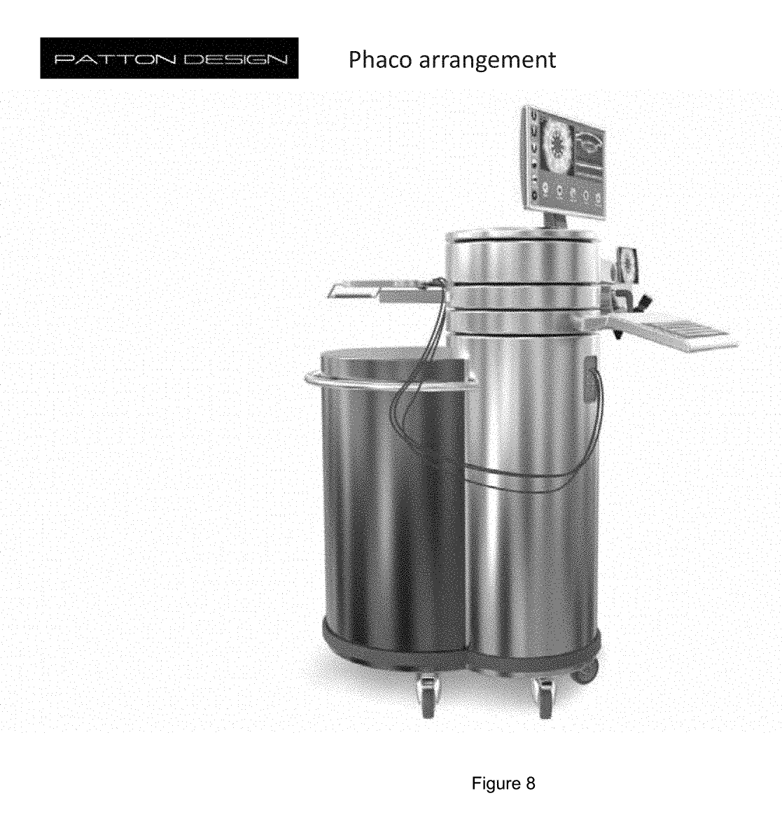

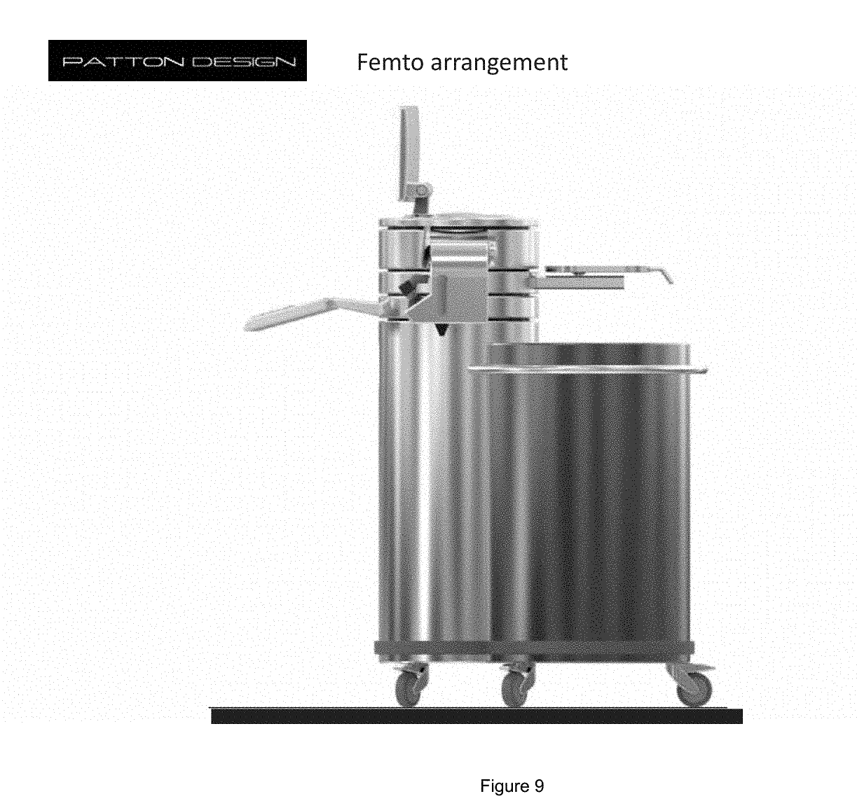

FIGS. 8-9 illustrate various views of one embodiment of the inventive subject matter in a phaco arrangement.

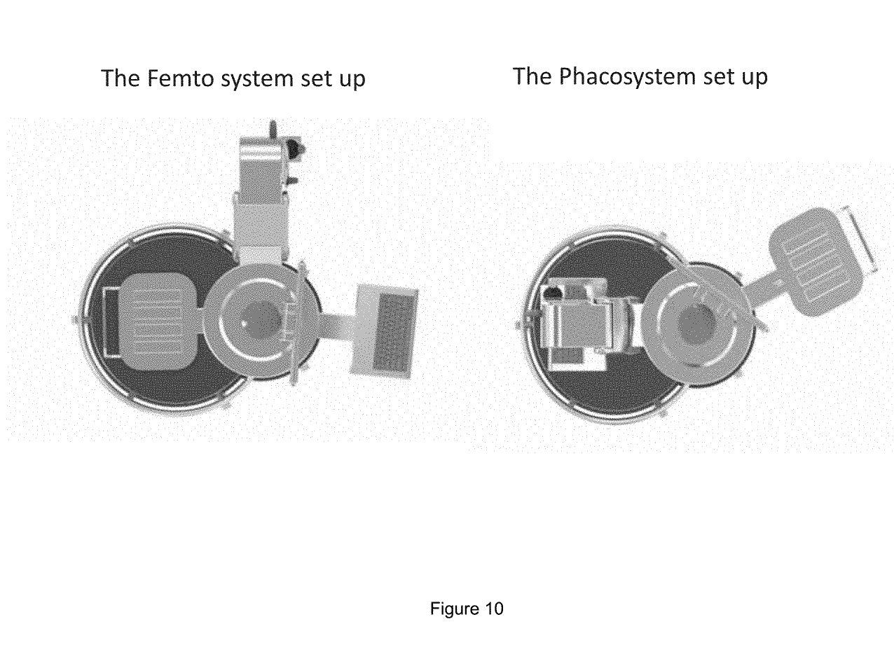

FIG. 10 illustrates top views showing the femto and phaco system set up.

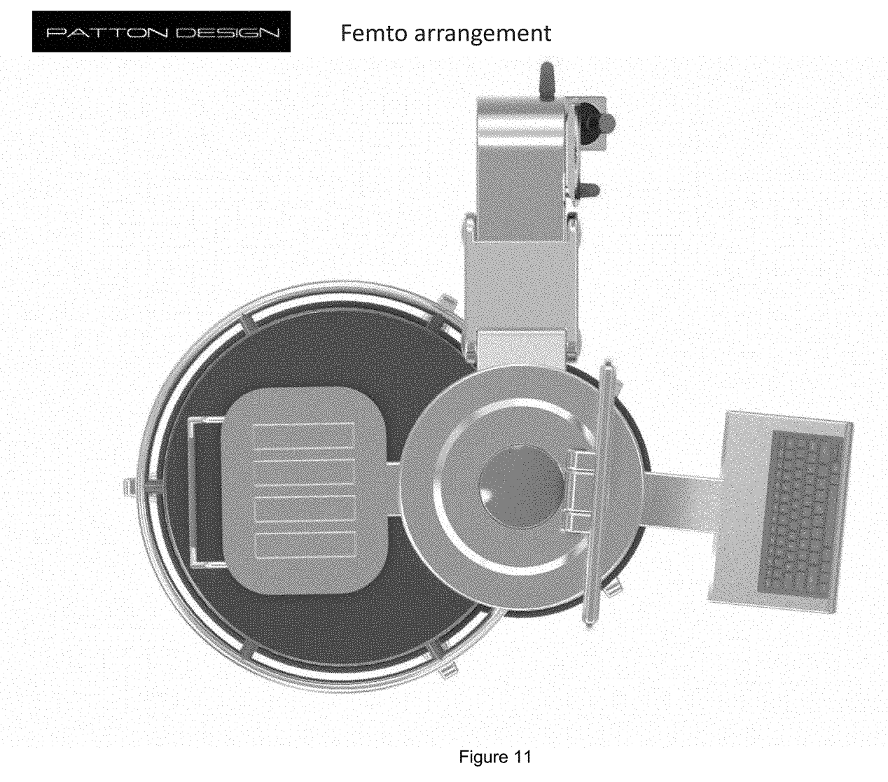

FIG. 11 illustrates a top view showing the femto arrangement.

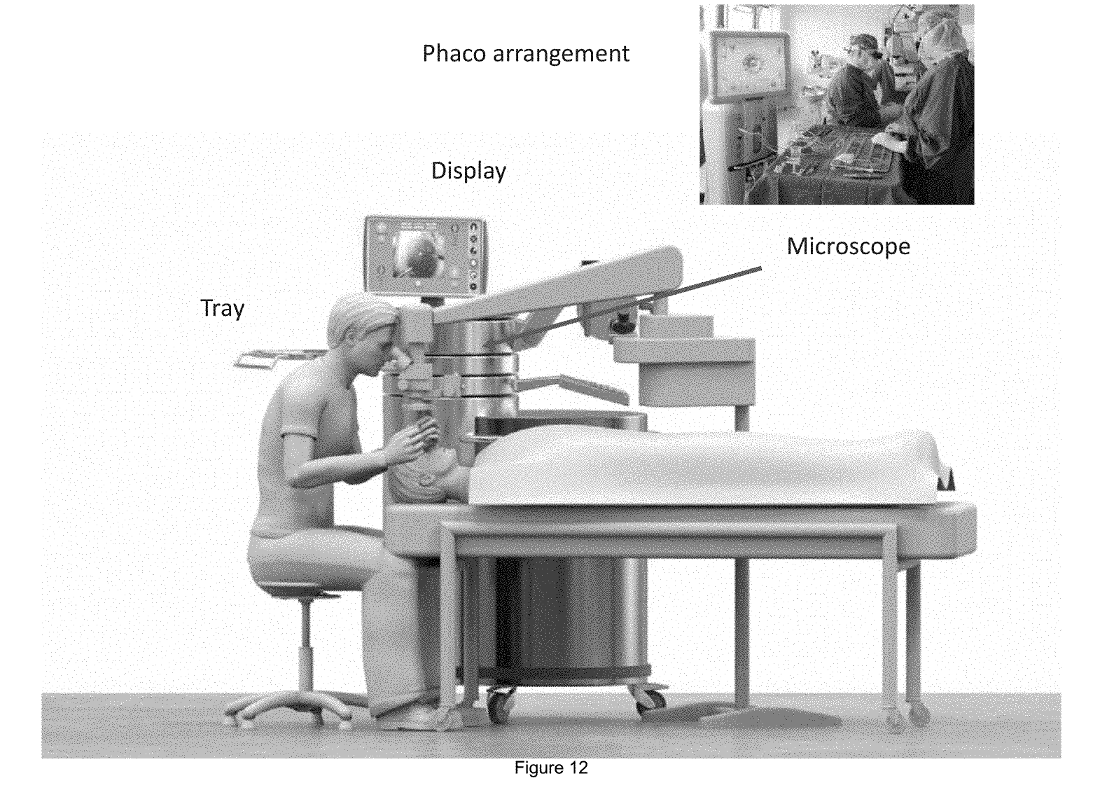

FIG. 12 illustrates a perspective view of the system in a phaco arrangement.

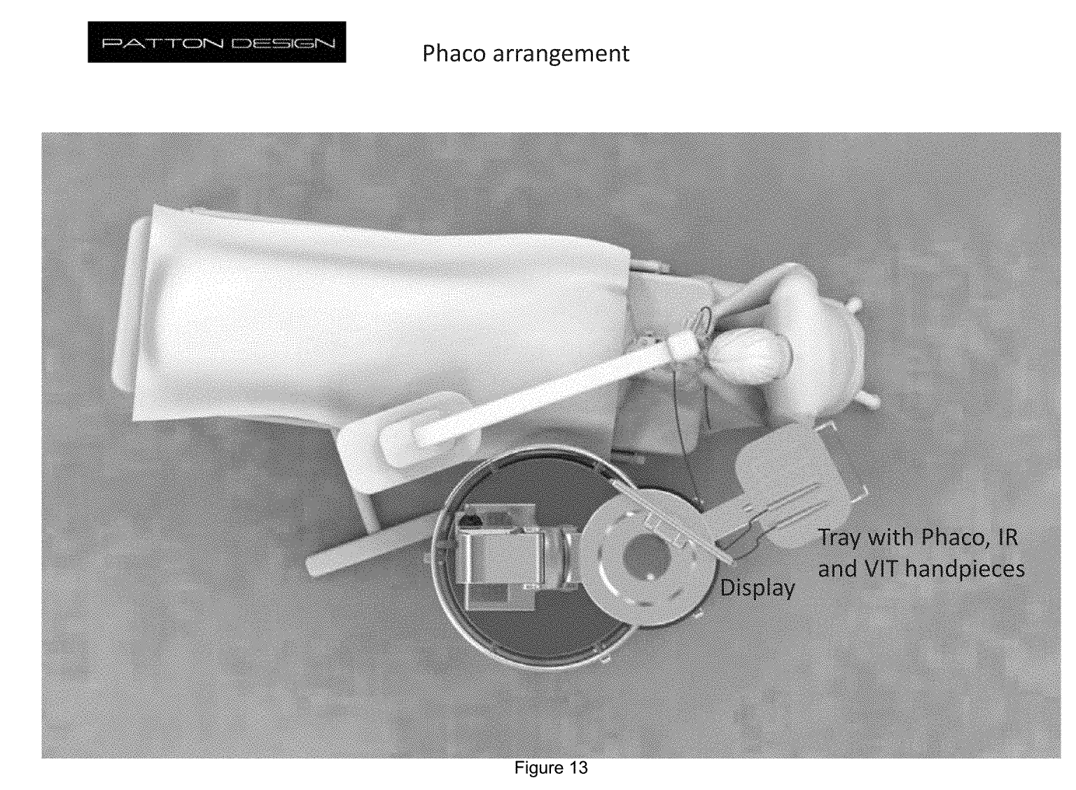

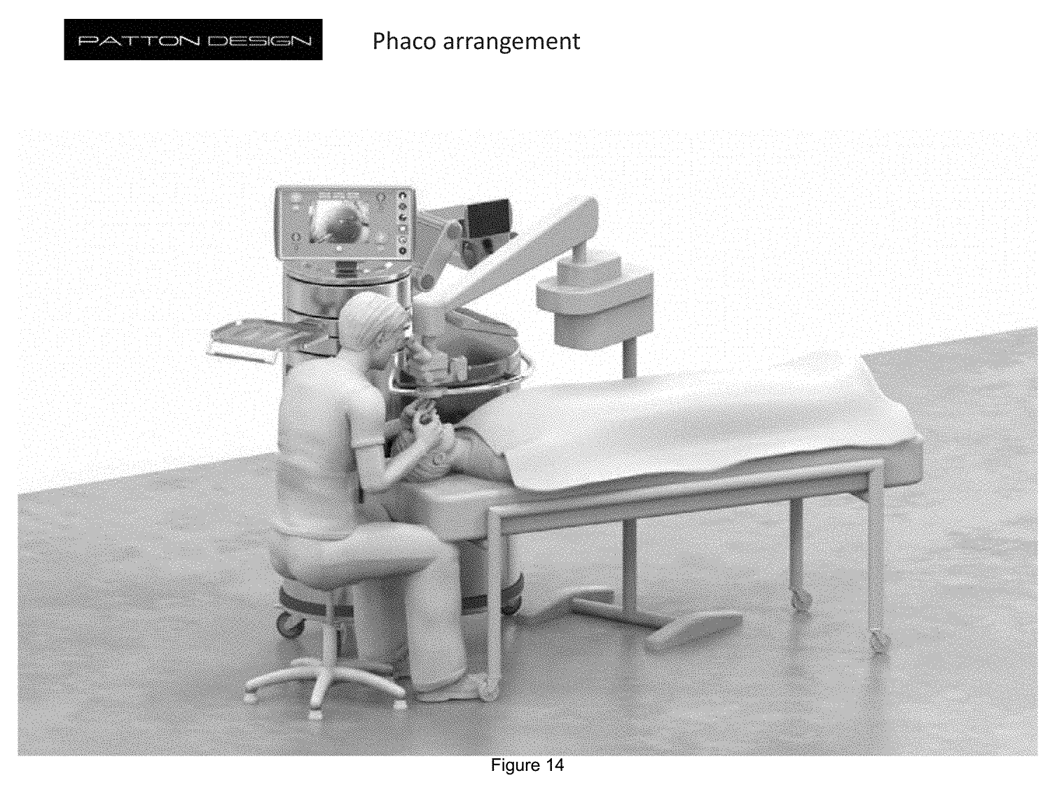

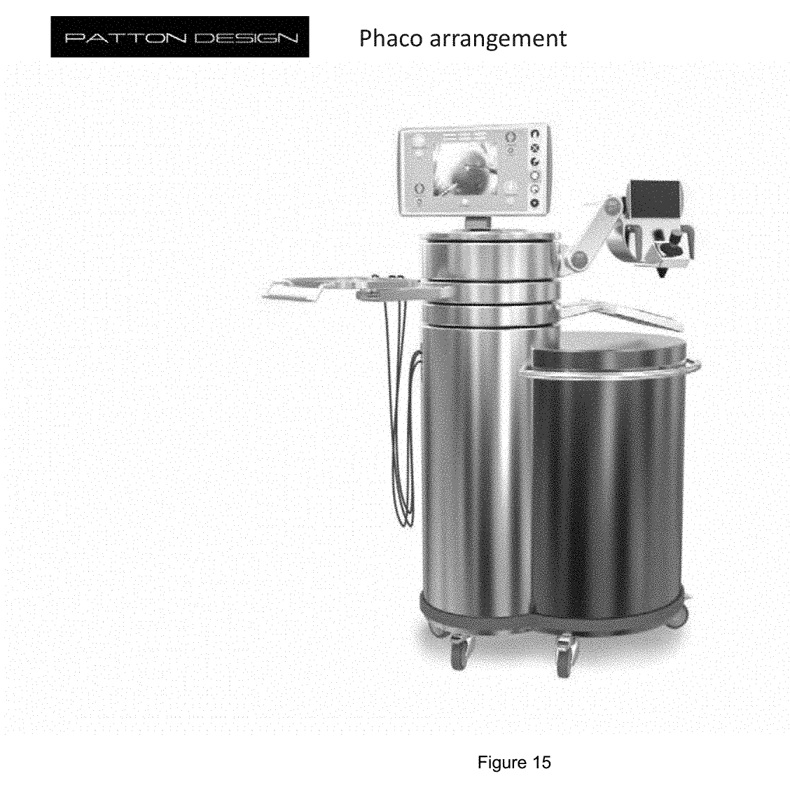

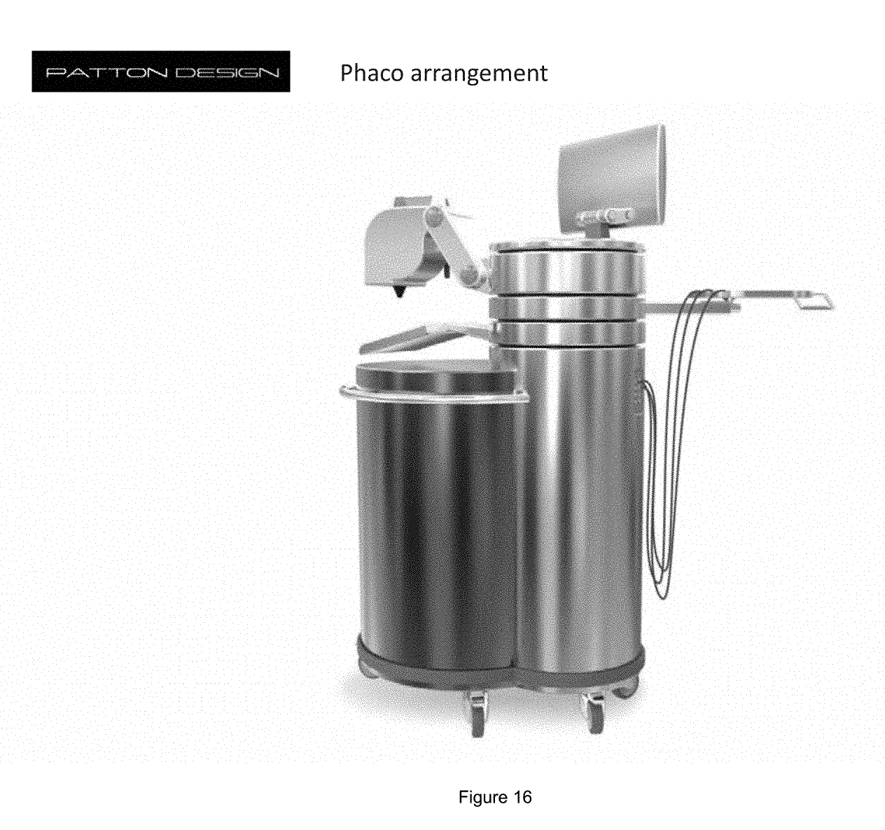

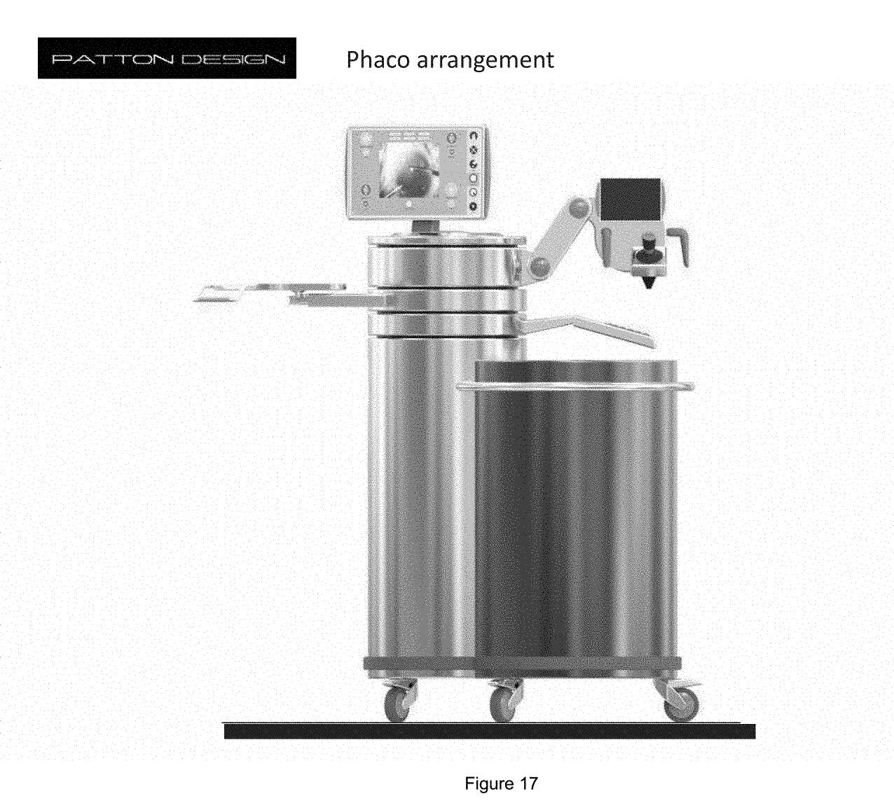

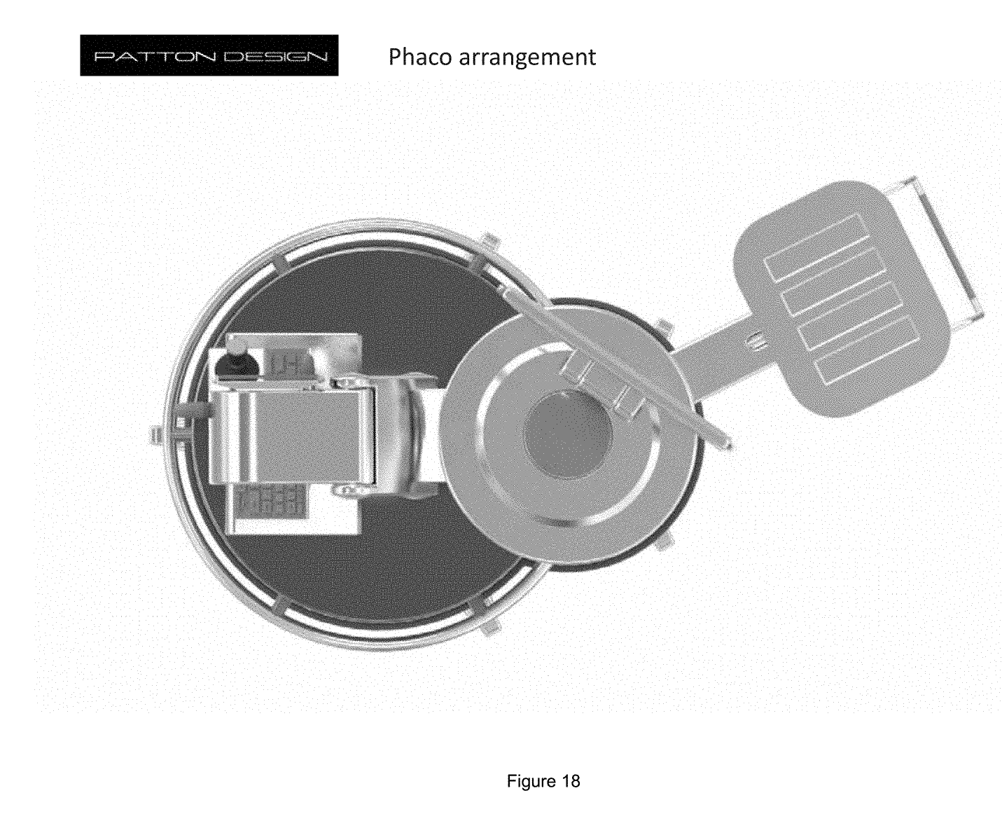

FIGS. 13-18 are various views of the device of FIG. 12 in the phaco arrangement.

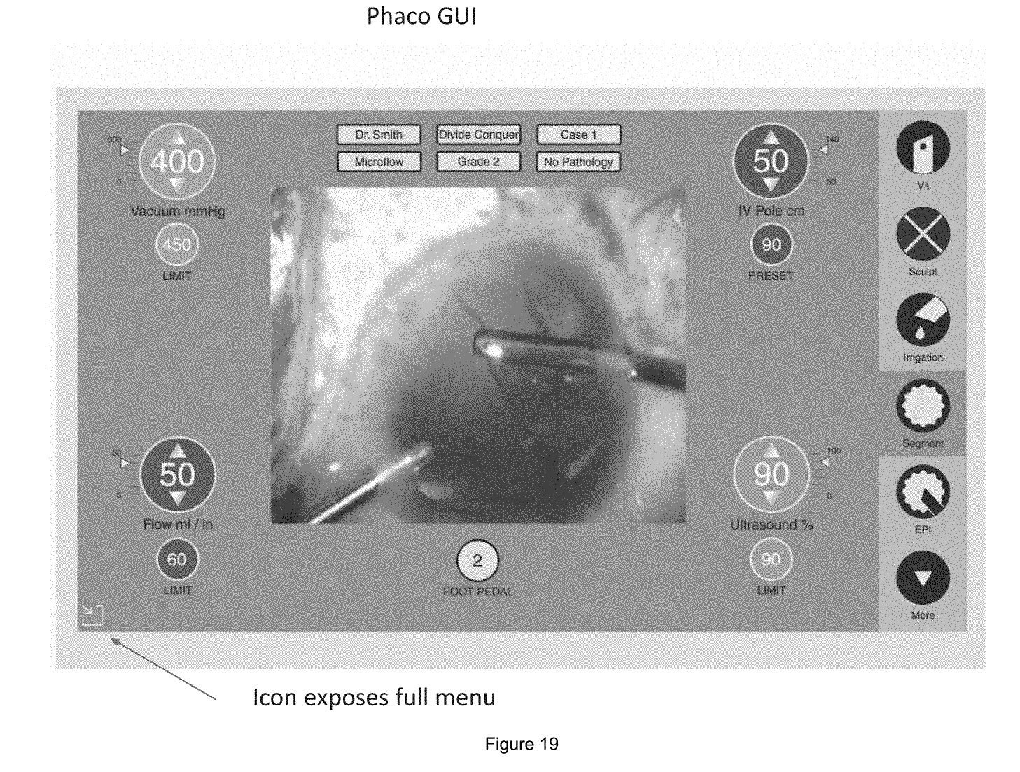

FIG. 19 is an exemplary embodiment of the graphical user interface for the phaco arrangement.

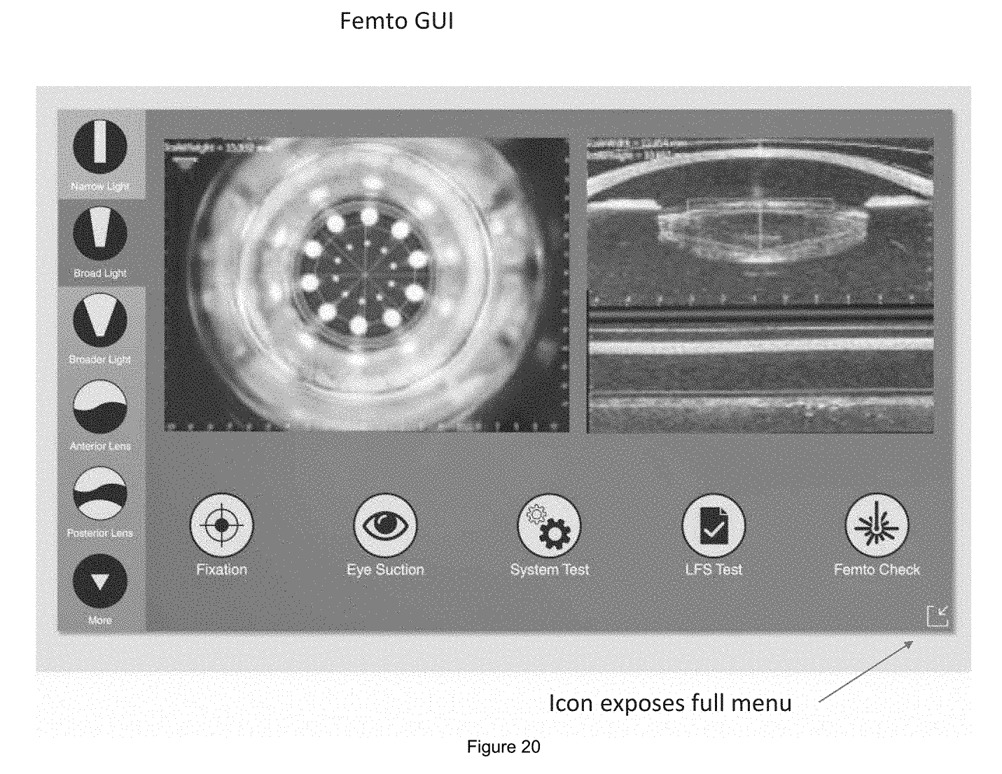

FIG. 20 is an exemplary embodiment of the graphical user interface for the femto arrangement.

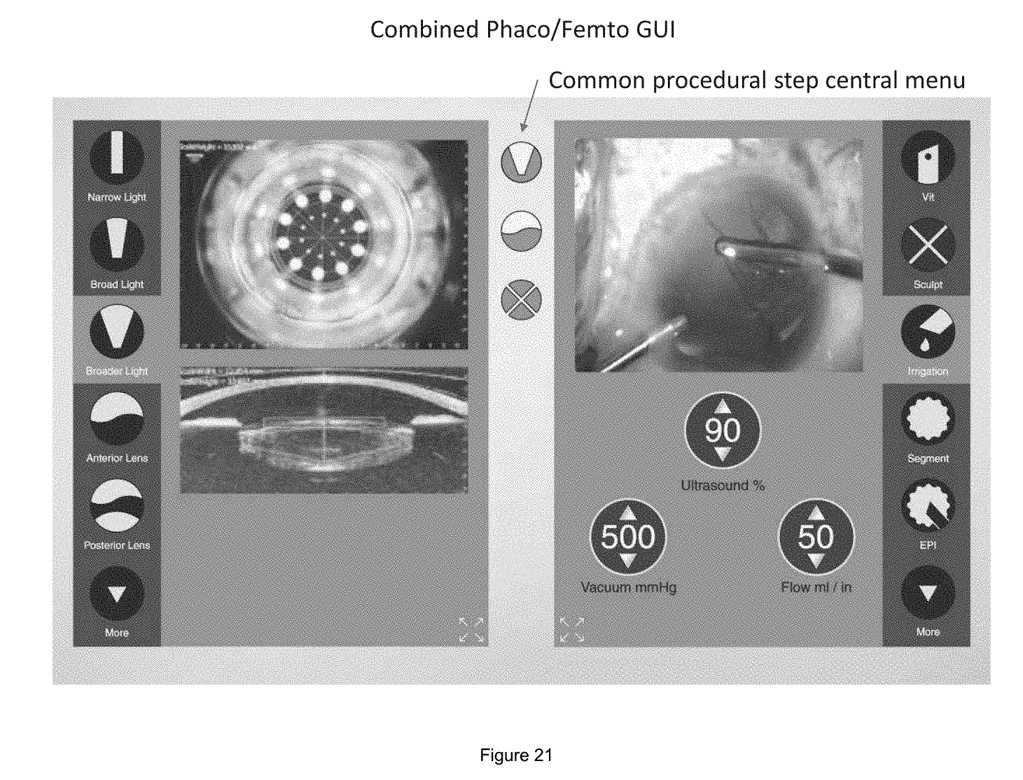

FIG. 21 is an exemplary embodiment of the graphical user interface for the combined phaco/femto setup.

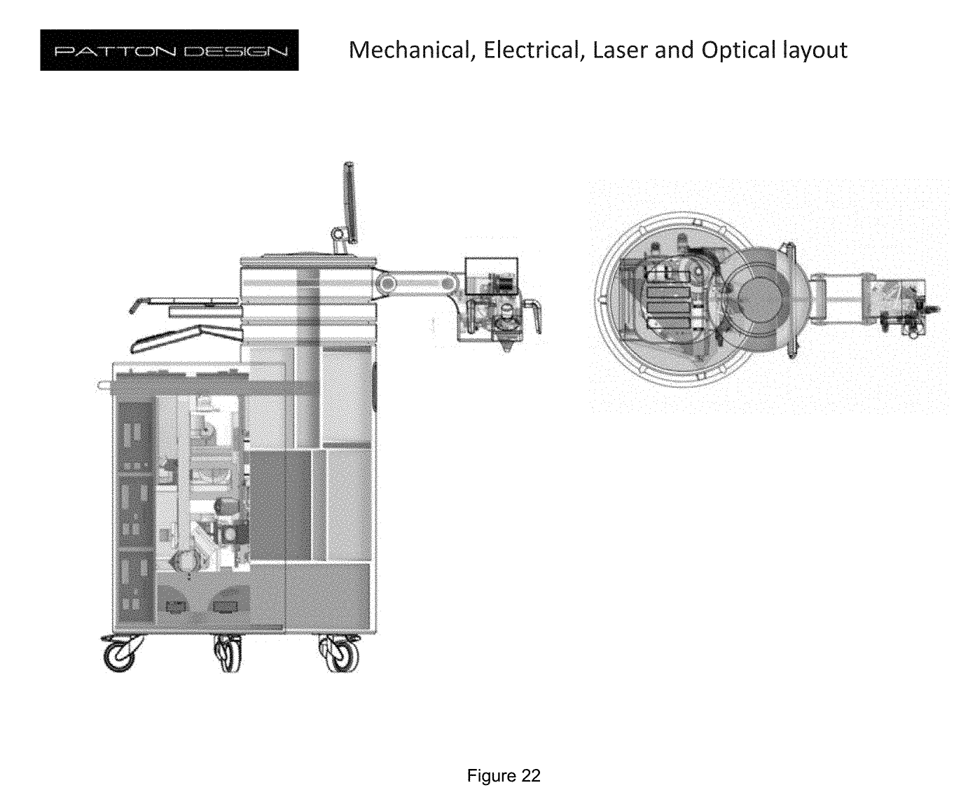

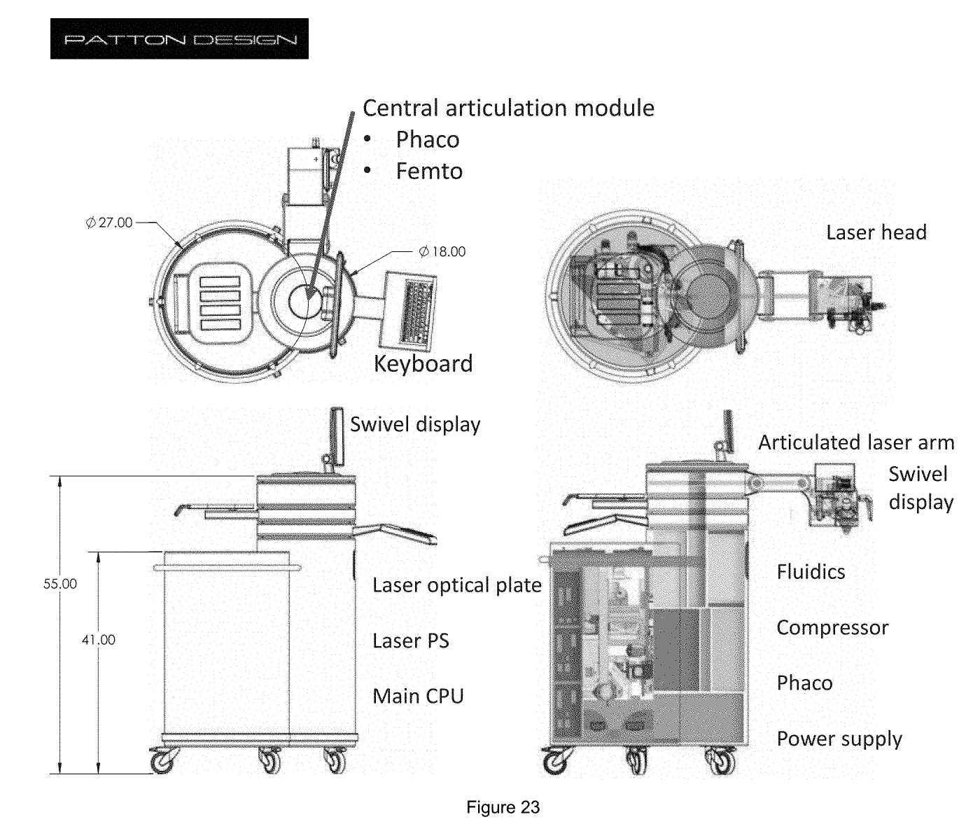

FIGS. 22-23 are schematics of one embodiment of the phaco/femto system.

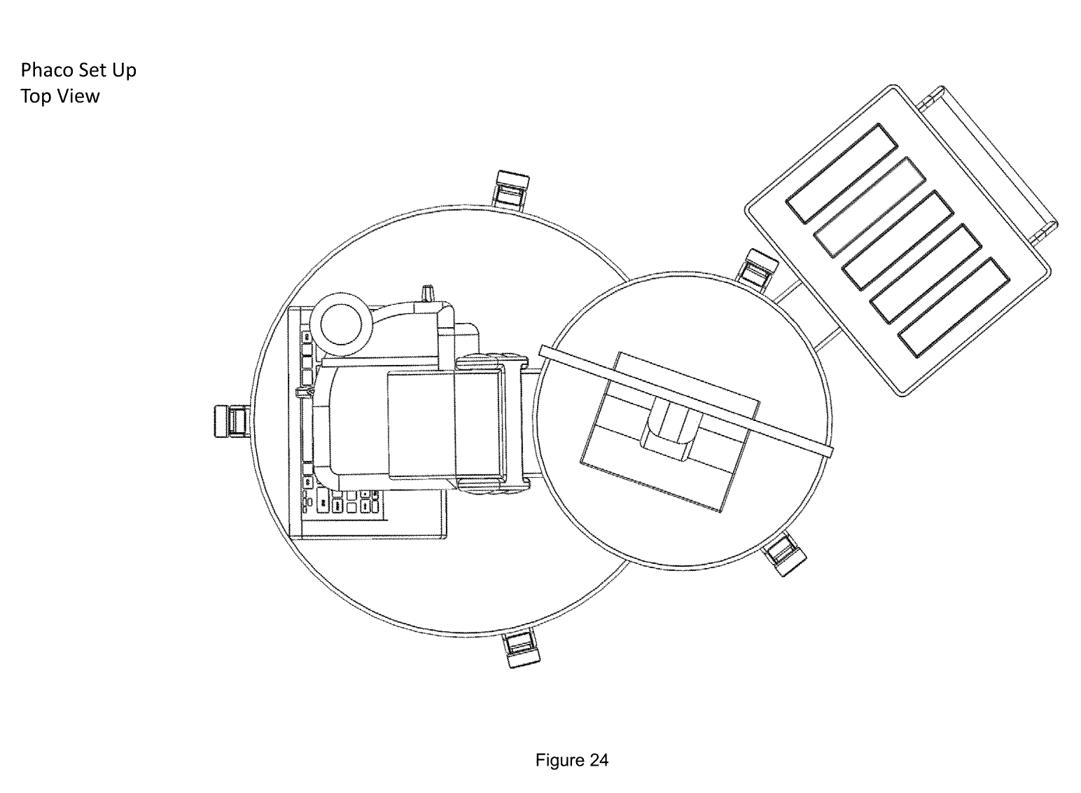

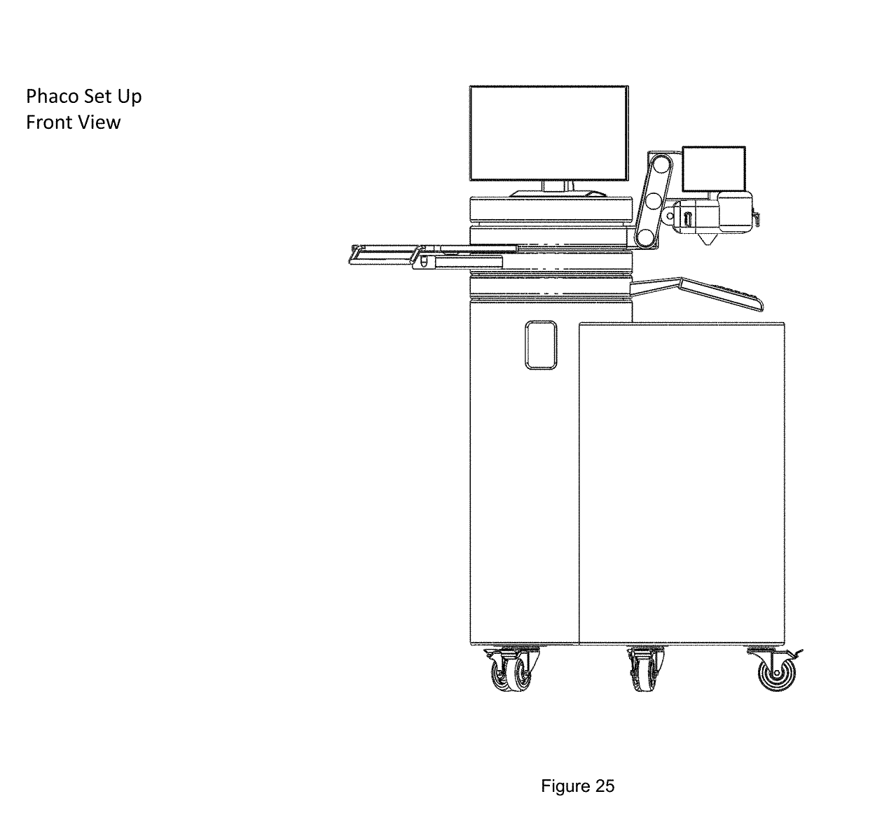

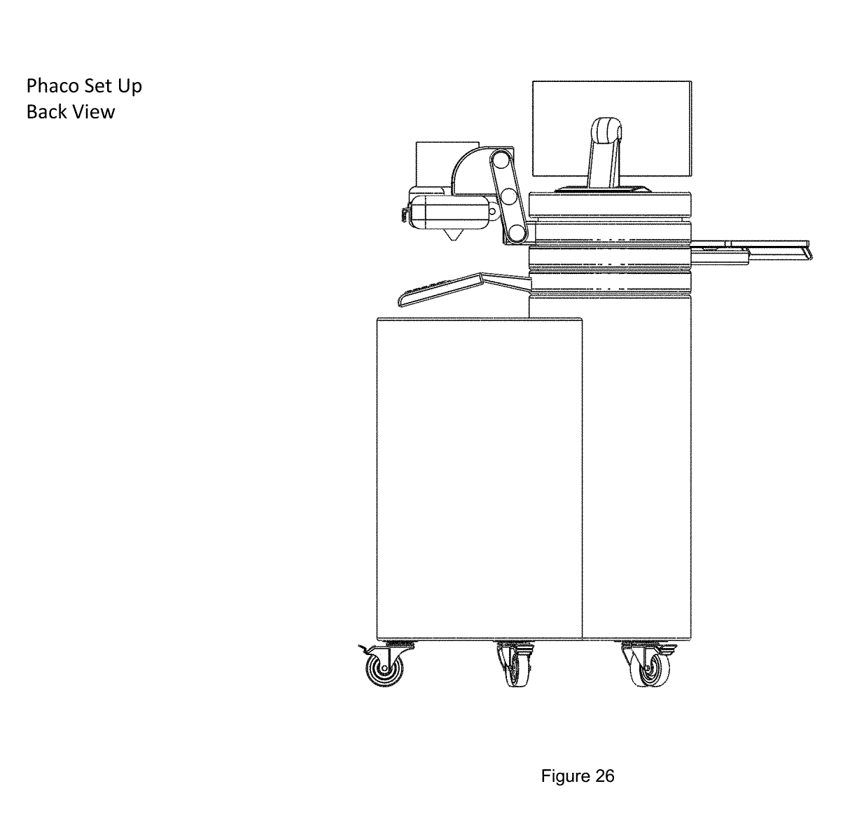

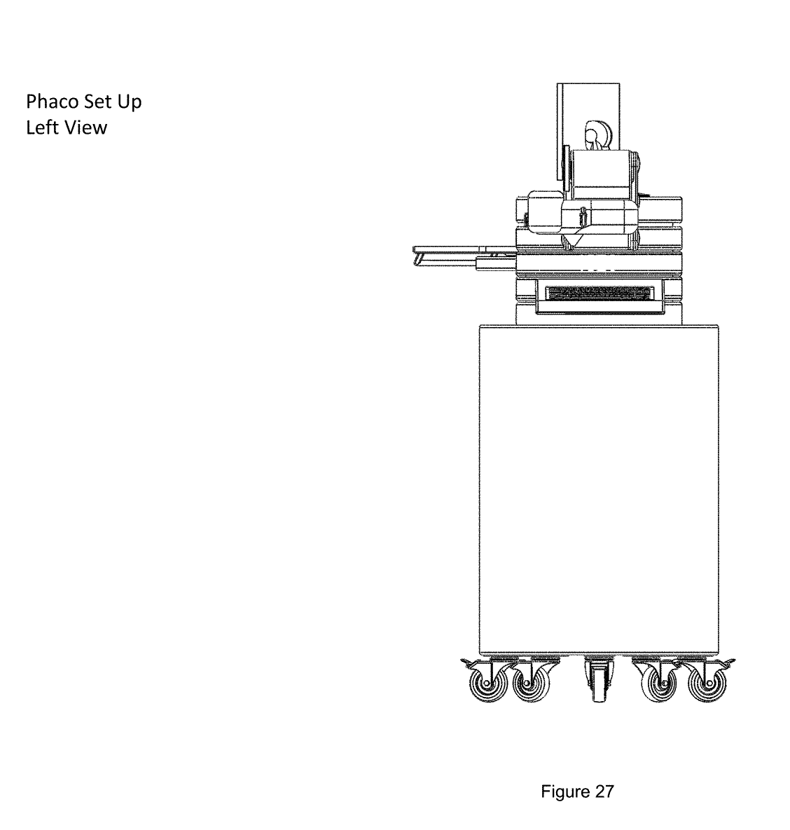

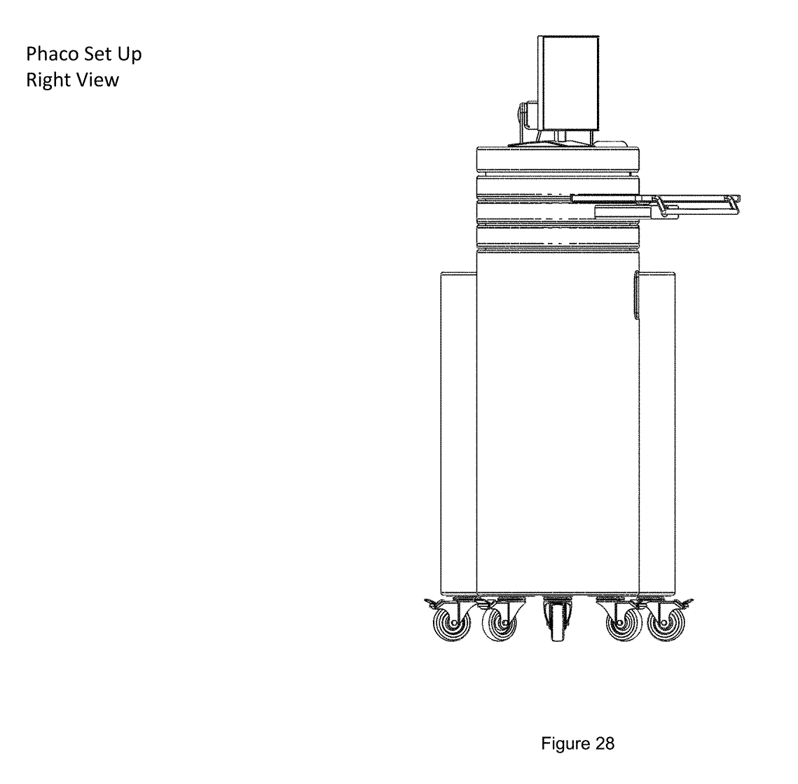

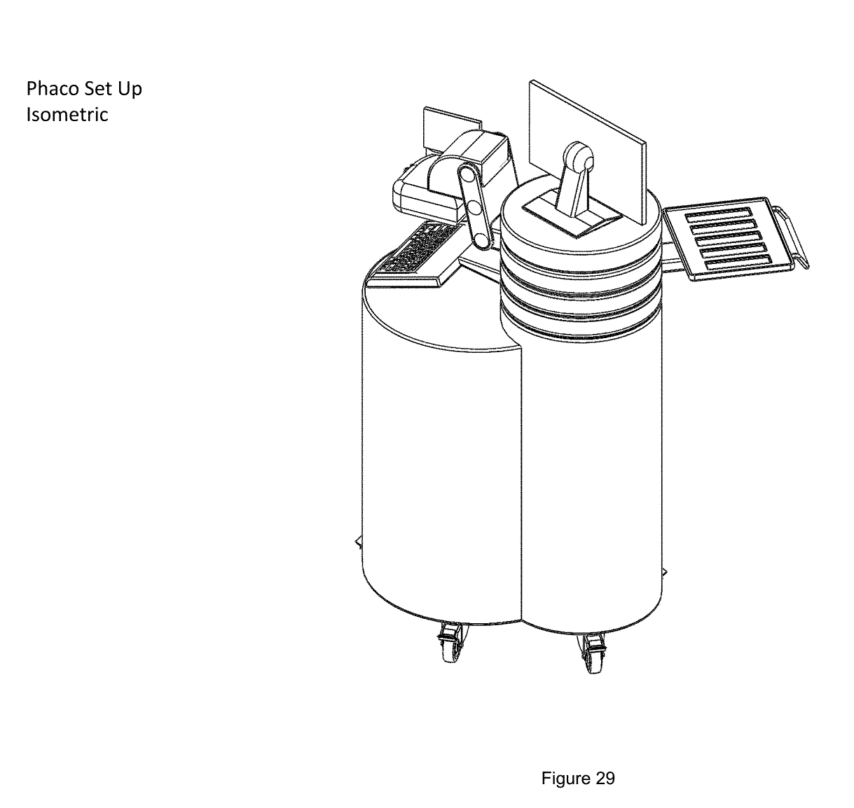

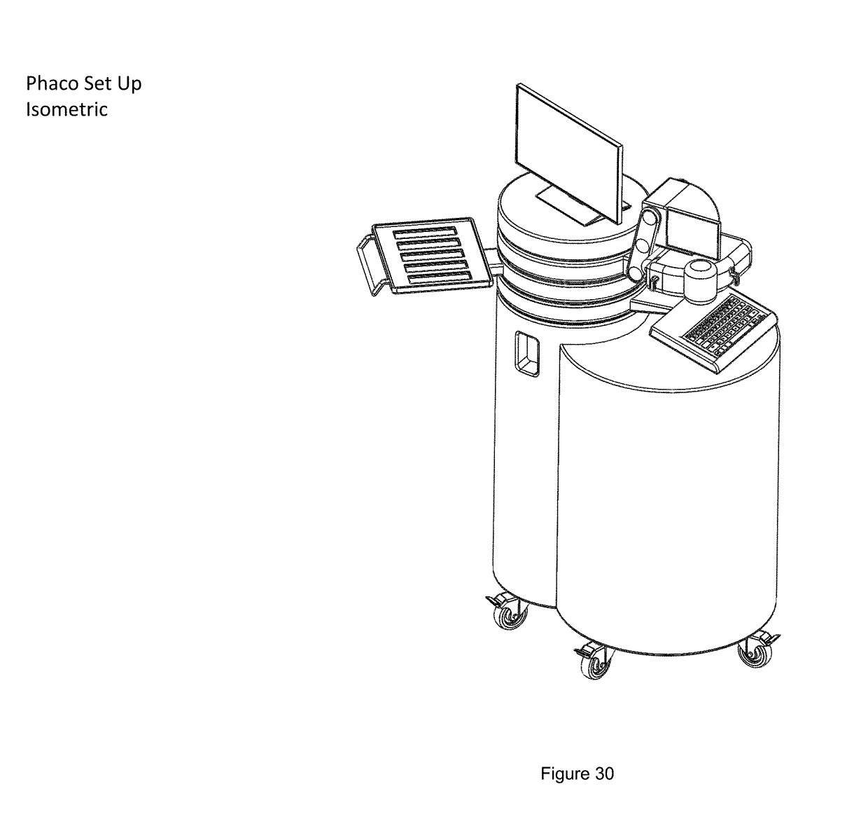

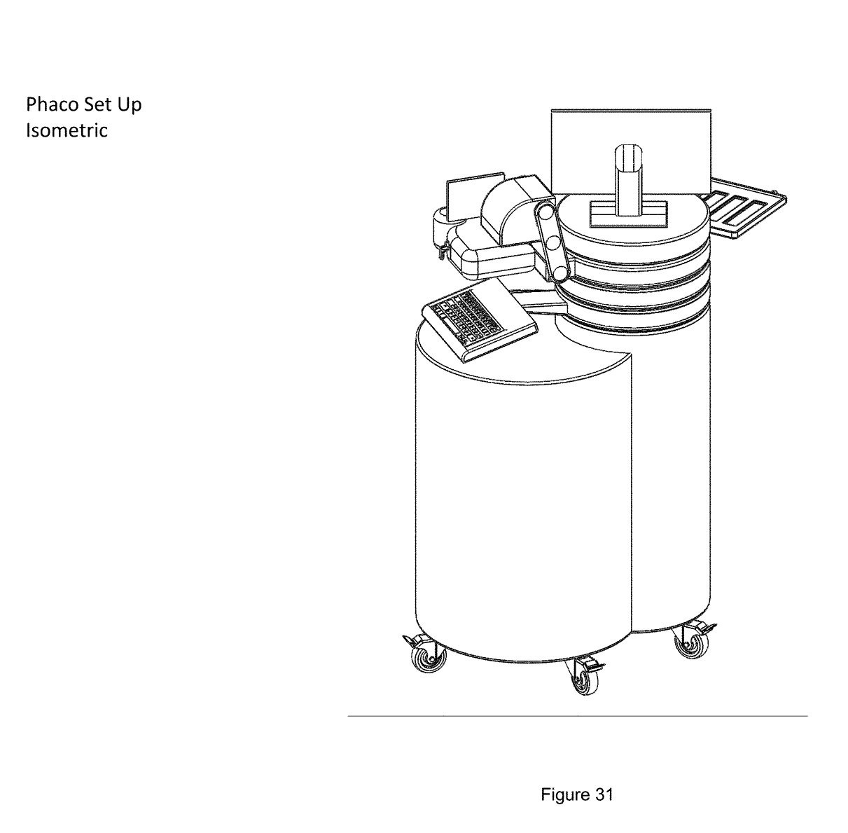

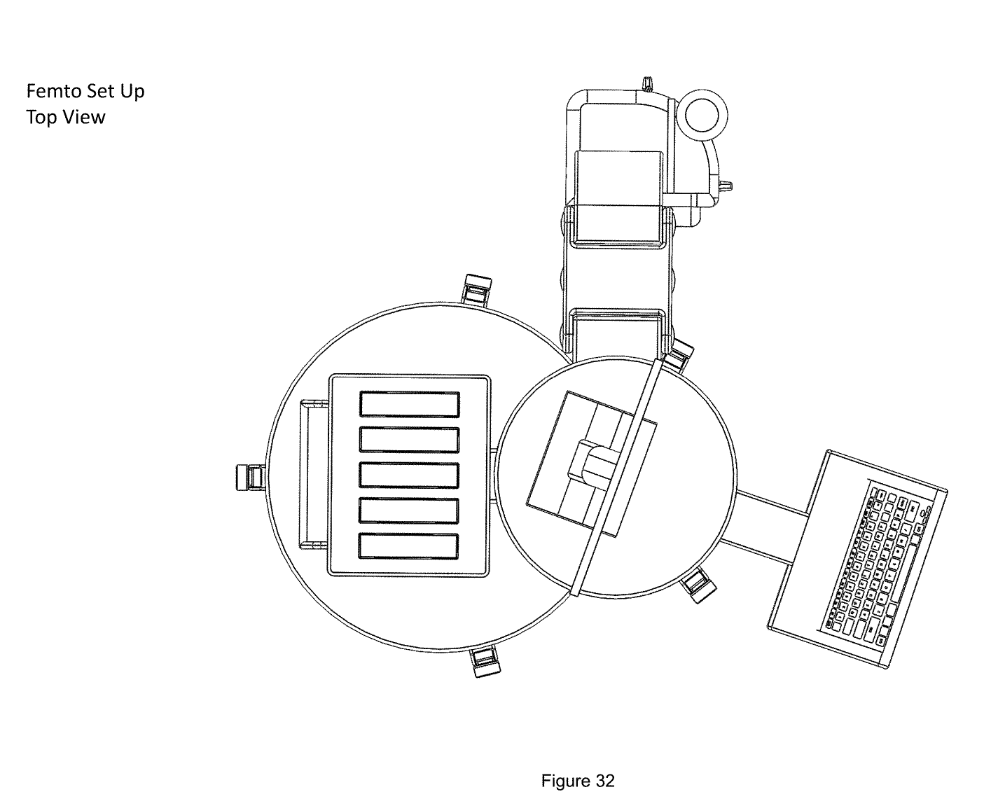

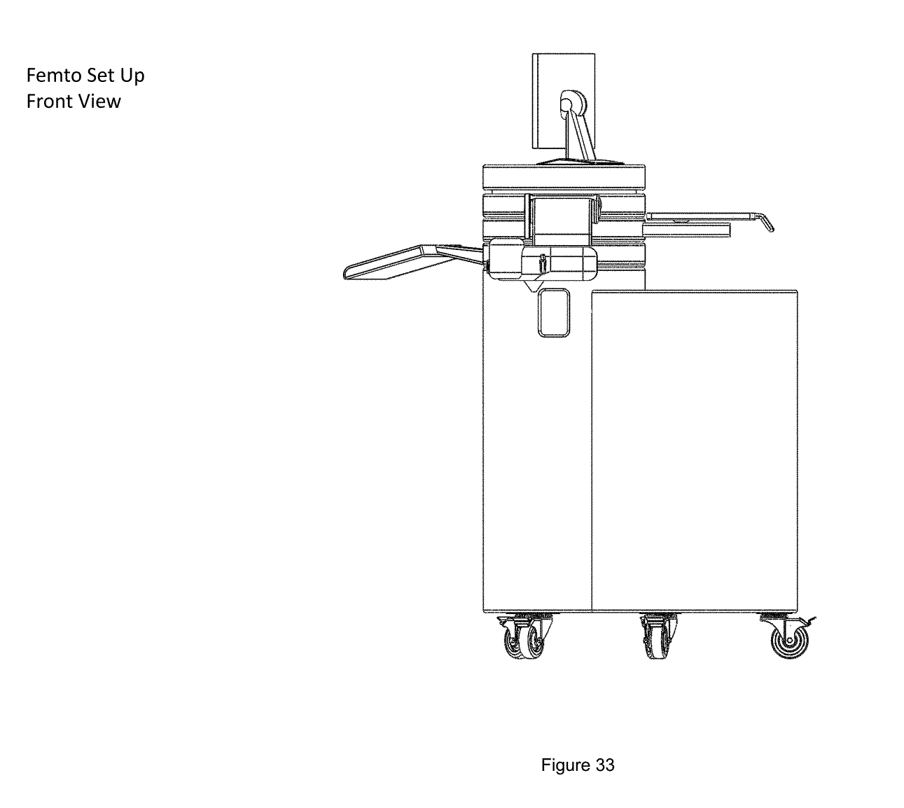

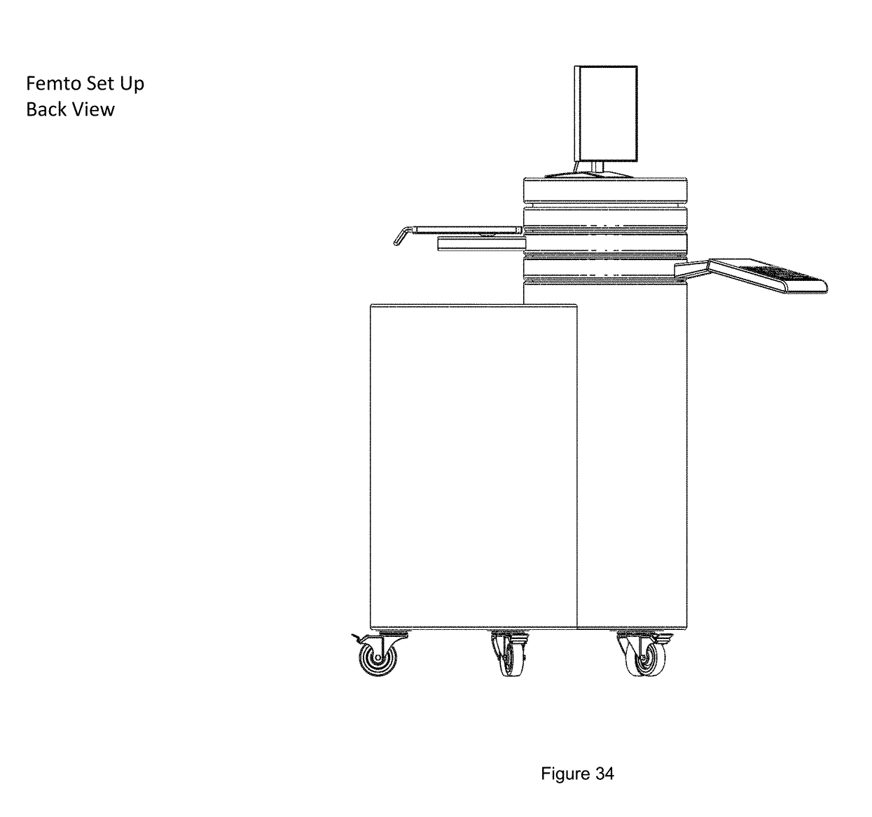

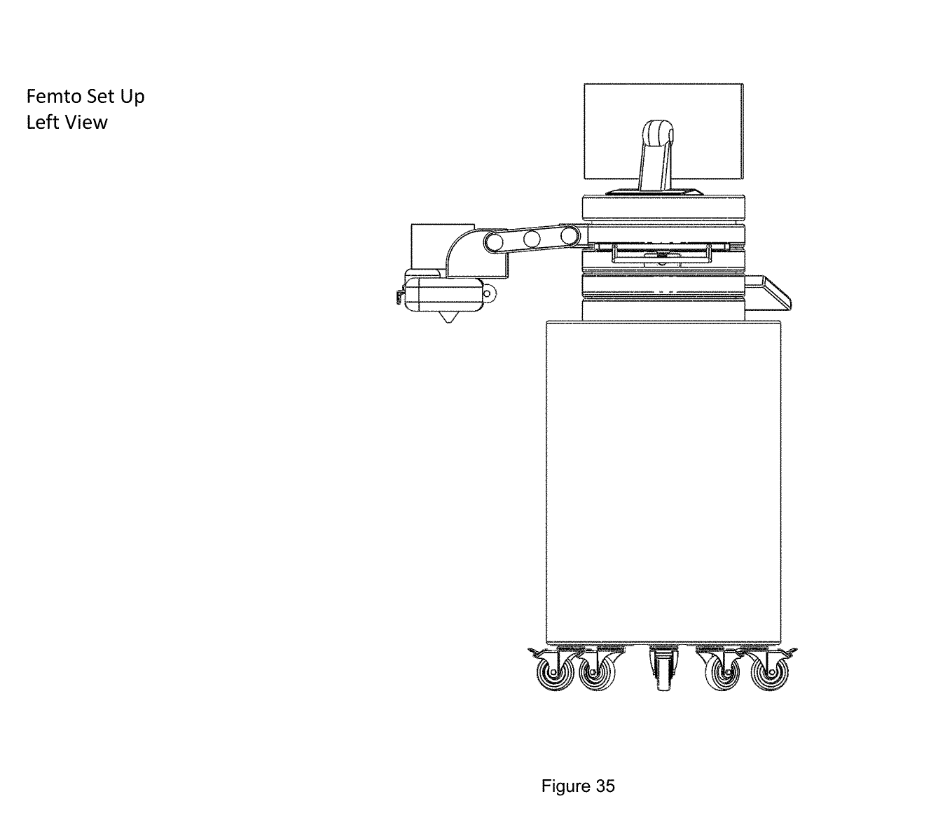

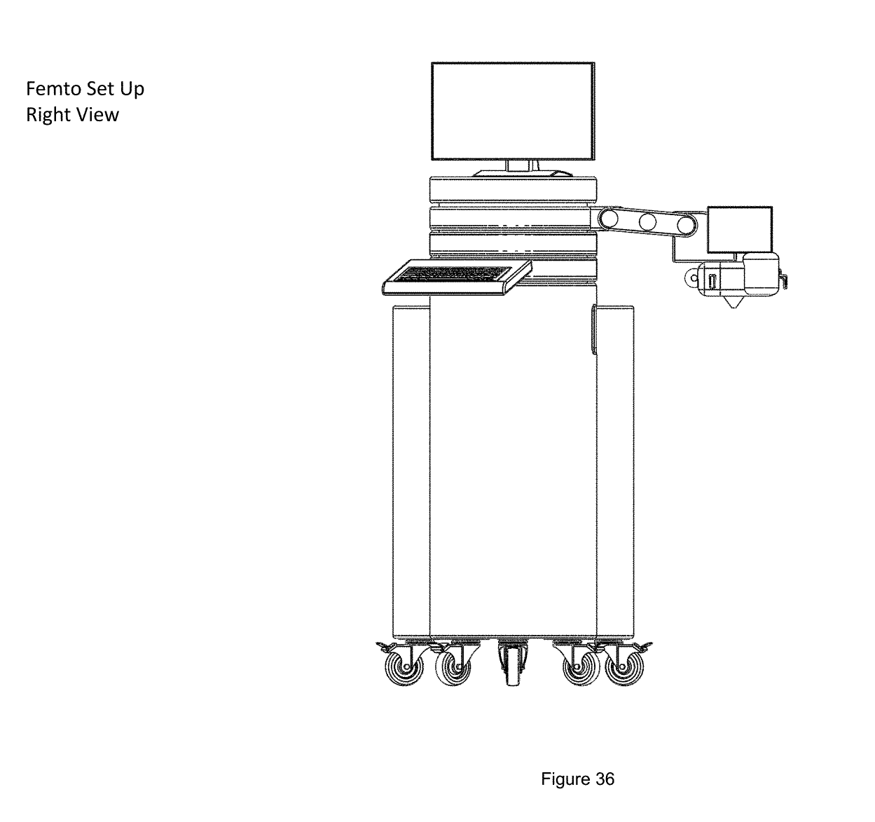

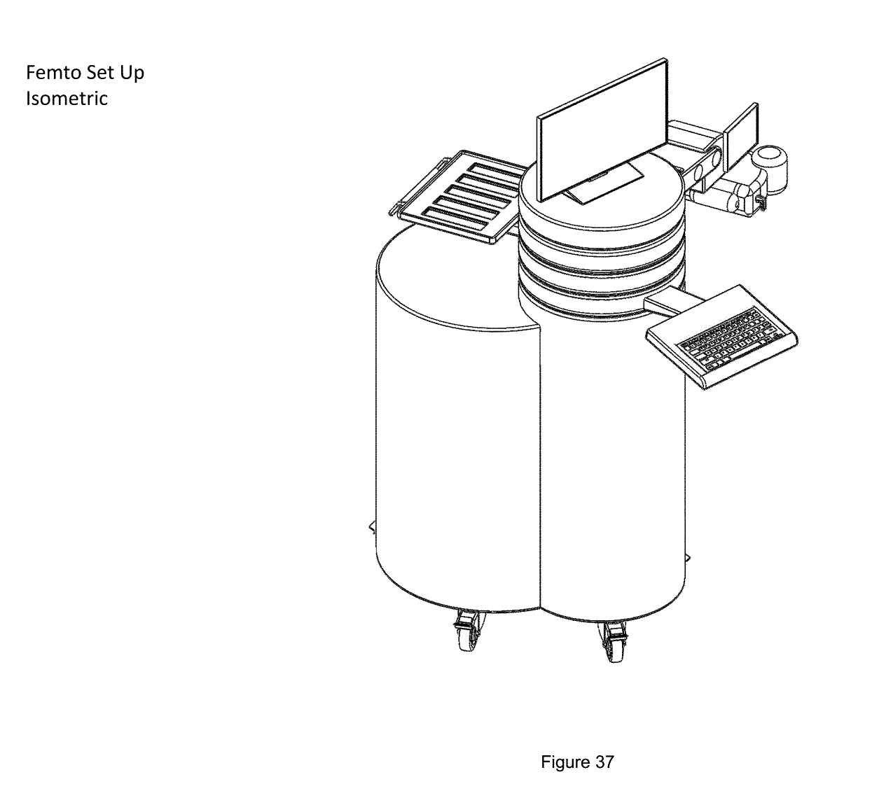

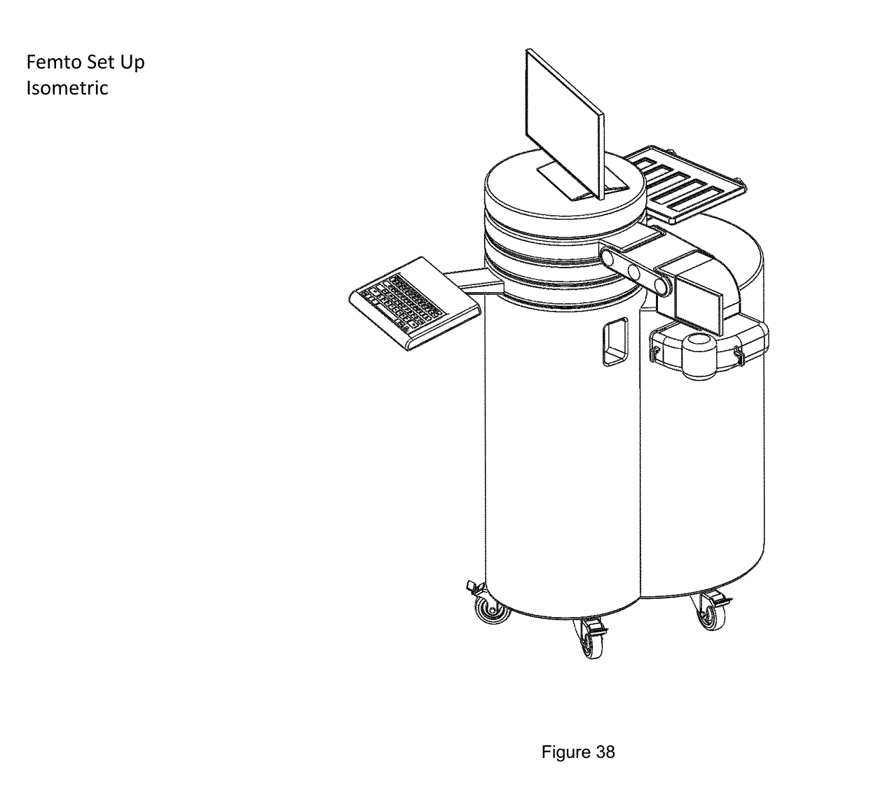

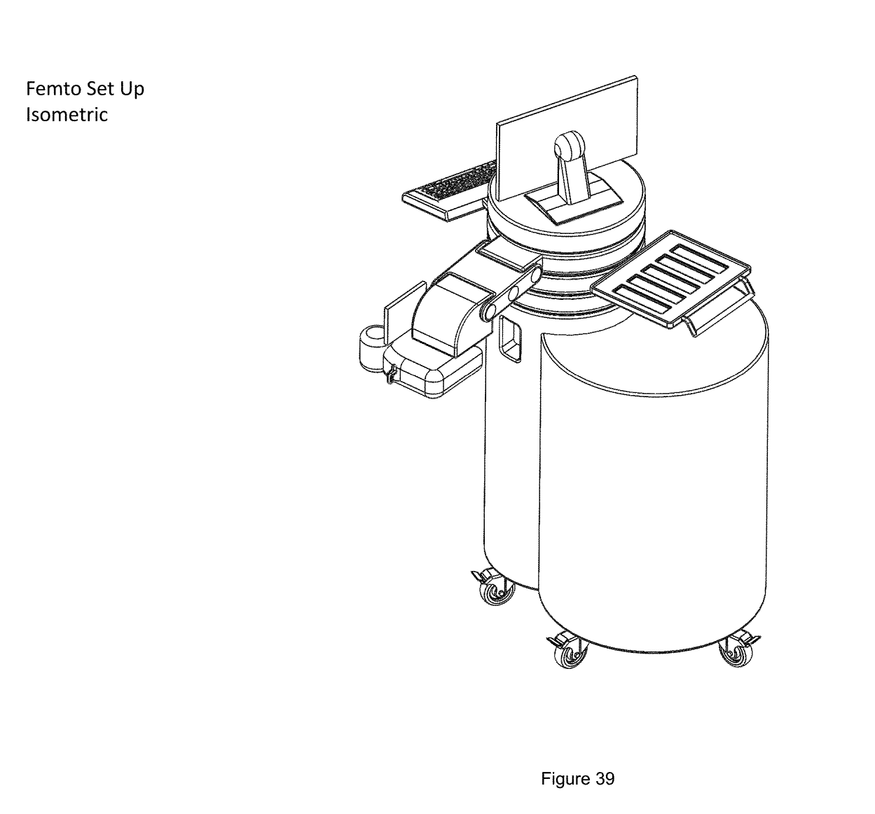

FIGS. 24-39 illustrate various views of another embodiment of the phaco/femto system shown in line drawings.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A system, comprising:
a housing;
wherein the housing comprises femto and phaco surgical systems and interfaces and a controller for controlling the femto and phaco surgical systems.

2. The system of claim 1, wherein the femto and phaco surgical systems can move and articulate with respect to each other that does not require moving a patient or surgeon from their positions before, during, or after each procedure.

3. The system of claim 1, wherein the controller permits a user to utilize both the femto and phaco surgical systems as required without moving a patient.

4. The system of claim 1, further comprising a femto laser head configured to move in a vertical X movement and horizontal Y movement or any angle to place the femto surface on a patient's eye or be removed from the patient's eye during surgery.

5. The system of claim 4, further comprising an alphanumeric keyboard configured to be articulated x/y or any angle with or independently from the femto optical head.

6. A combined femto/phaco system, comprising:
a phaco tray configured to hold the phaco, VIT, irrigation and aspiration surgical handpieces;
wherein the tray is configured to articulate along x and y axes or at any angle such that the tray can be placed into the surgical field at or about patient eye height for a phaco surgical procedure without requiring movement of the patient or surgeon.

7. The system of claim 1, further comprising a microscope integrated into the housing that is movable along x and y axes or at any angle such that the microscope can move in or out of position for a phaco procedure.

8. The system of claim 7, wherein the femto and phaco surgical systems and the microscope are each configured to articulate from a central column to permit articulation from a central point and allow the femto and phaco surgical systems and/or the microscope to be placed into surgical position before, during and after surgery without requiring moving of a patient.

9. The system of claim 8, wherein the articulation is circular in the horizontal.

10. The system of claim 9, wherein the femto and phaco surgical systems and the microscope can also extend and retract in a vertical movement and can extend or retract and at any angle.
